(19) United States
(12) Patent Application Publication
(10) Pub. No.: US 2003/0100931 A1
(43) Pub. Date: May 29, 2003

(54) Brain signal feedback for pain management

(76) Inventor: Keith Mullett, Valkenburg ann de Gaul (NL)

Correspondence Address: MEDTRONIC, INC., 710 MEDTRONIC PARKWAY NE, MS-LC340, MINNEAPOLIS, MN, 55432-5604, US

(21) Appl. No.: 10/132,508

(22) Filed: Apr. 26, 2002

Related U.S. Application Data
(60) Provisional application No. 60/333,473, filed on Nov. 28, 2001.

(51) Int. Cl. A61N 1/34

(52) U.S. Cl. 607/46

(57) ABSTRACT

System and method for pain management therapy that provides customized signal analysis for each patient's perception of pain. In addition, the pain relief response to the sensed brain wave signals indicating perceived pain can also be customized for each patient. An implantable medical device system senses and analyzes a brain wave signal for an indication of pain. Feedback control for providing pain management therapy with the implantable medical device system is provided based on the indication of pain from the analysis of the brain wave signal. The brain wave signal can continue to be sensed and analyzed to determine whether the pain management therapy is effective in reducing or eliminating the indication of pain in the brain wave signal. Based on the analysis, parameters used to control the amount of pain management therapy and/or the type of pain management therapy can be adjusted.

This application claims the benefit of U.S. Provisional Application Serial No. 60/333,473, filed Nov. 28, 2001, which is incorporated herein by reference in its entirety.

### FIELD OF THE INVENTION

This invention relates to pain management, and more particularly to feedback control systems and techniques for pain management.

### BACKGROUND

Pain manifests itself in many forms. Pain can be associated with actual or potential tissue damage. Examples include pain caused by an illness, an injury, or as a result of a medical procedure. Pain can also be associated with unknown causes. One example where unknown causes might cause pain is a headache. Tension headaches and migraine headaches account for the vast majority of all headaches. Migraine headaches have a complex of symptoms that can include discrete episodes of severe headaches with associated features, such as phonophobia, photophobia, nausea, and emesis.

A second example is Chronic Post Stroke Pain (CPSP). CPSP is caused by tissue damage in the brain, but is perceived by the individual as pain in specific parts of the body. This pain is characterized by an intolerable sensitivity to touch and temperature in an otherwise numb part of the body. CPSP is often described as a burning sensation. This pain often presents three to six months after the stroke.

Pain may be mediated by specific nerve fibers to the brain where its conscious appreciation may be modified by various factors. The specific pain nerve fibers can be stimulated through pain receptors. Pain receptors are specific for different types of potentially harmful conditions. For example, pain receptors can be sensitive to mechanical forces, temperature, and chemical changes in and to the body. Once stimulated, the receptor produces an electrical signal that is sent to the brain, where the stimulation is sensed as a pain.

Pain may also be a result of damage or injury to a peripheral nerve, a region of the spinal cord, or the brain. These injuries may result from a stroke, a head injury, a spinal injury, a bulge in a spinal disk, or a projectile impinging on the nervous system. Such injury often results in a pain perceived remotely from the point of injury and may be due to change in the spontaneous firing rate of neurons.

Pain is classified as either acute or chronic. Various diseases and/or disorders can cause chronic pain. Chronic pain continues for a month or more beyond the usual recovery period for an illness or an injury. In addition, chronic pain can be present over months or years as a result of a chronic condition. The chronic pain can be continuous or episodal.

Acute pain is often the result of injury or surgery. In contrast to chronic pain, acute pain is typically ameliorated with treatment or through the body's own healing powers. Acute pain often causes vital signs (pulse, respiration, blood pressure) to change from normal and is usually accompanied by an expressed longing (i.e., “I can't wait to get well”). In contrast, chronic pain patients often show flat affect or depressive symptoms and no change in vital signs because their system has adjusted to the pain both physiologically and emotionally.

Pain management is a large goal in treating both acute and chronic pain. Through pain management, the physician hopes to eliminate pain in the patient, or at least modulate it to a level of pain that no longer presents bothersome effects for the patient. Although pharmacological agents are used for both acute and chronic pain, pharmacological treatment is often not successful in relieving pain. In addition, side effects from the pain medications can affect the quality of the patient's life. As will be appreciated, it is also important to allow patients to be active and functional while the pain is being managed.

Electronic systems for pain management have also been suggested. Table 1 lists documents that disclose systems and methods for pain management.

TABLE 1

| Patent | | |
|---|---|---|
| Number | Inventors | Title |
| 5,653,739 | Maurer et al. | Electronic Pain Feedback System and |
| | | Method |
| 5,938,690 | Law et al. | Pain Management System and Method |
| 6,161,044 | Silverstone | Method and Apparatus for Treating |
| | | Chronic Pain Syndromes, Tremor, Dementia |
| | | and Related Disorders and for Inducing |
| | | Electroanesthesia Using High Frequency, |
| | | High Intensity Transcutaneous Electrical |
| | | Nerve Stimulation. |
| 6,308,102 | Sieracki et al. | Patient Interactive Neurostimulation System |
| | | and Method |

These systems, however, require patient feedback information for assessing and treating pain on a regular basis, and/or are indicated for only a limited number of pain producing conditions. Relying on patient feedback information for treating pain can lead to situations where the patient provides more pain relief therapy than is actually required. This situation occurs when the patient believes that if a little therapy is good, then more is better. As a result, the patient's body adapts to the over-stimulation, which in turn requires the patient to use ever increasing levels of therapy. Therefore, a need for more efficient, effective, and generalized treatments of pain management continues to exist.

All documents listed in Table 1 above are hereby incorporated by reference herein in their respective entireties. As those of ordinary skill in the art will appreciate readily upon reading the Summary of the Invention, Detailed Description of the Preferred Embodiments and Claims set forth below, many of the devices and methods disclosed in the patents of Table 1 may be modified advantageously by using the techniques of the present invention. In addition, providing the documents listed in Table 1, or elsewhere in this document, is not an admission that the cited document is prior art to the present invention.

### SUMMARY OF THE INVENTION

The present invention has certain objects. That is, various embodiments of the present invention provide solutions to one or more problems existing in the prior art with respect to pain management, and with respect to feedback control systems and techniques for pain management in particular. Such problems include, for example, requiring patient feedback on a regular basis to ensure effective pain management therapy, pain management systems whose application is limited to only a select number of pain producing conditions, the present inability to automatically control an implantable medical device system for providing pain management based on brain wave signals sensed by the implantable medical device system. Various embodiments of the present invention have the object of solving at least one of the foregoing problems.

In comparison to known implementations of providing pain management, various embodiments of the present invention may provide one or more of the following advantages: using an implantable medical device system to identify defined patterns in a sensed brain signal that indicate the presence of pain; providing pain management therapy from an implantable medical device system based on the presence of pain from the analysis of the brain wave signals; and providing automatic feedback control to the implantable medical device system for pain control in a patient.

Objects of the present invention overcome at least some of the disadvantages of the foregoing systems by providing a system and method that sense and analyze brain wave signals for the occurrence of pain. In one example, the present invention provides a system and method of automatic signal analysis to identify the occurrence of pain in a patient. In an additional example, the present invention provides an implantable system and method of sensing and analyzing brain wave signals for the occurrence of pain and providing pain management therapy when the occurrence of pain is sensed.

In addition, the invention provides an implantable system and method for sensing and analyzing brain wave signals for the occurrence of pain and providing feedback control of the implantable system based on the analysis of the brain wave signal. Furthermore, the invention provides an implantable system and method for automatically regulating the application of pain management therapy. Also, the invention provides an implantable system and method for reducing the amount of time in applying pain management therapy. The invention also conserves depletable resources of an implantable system for pain management through feedback control and regulation of the application of pain management therapy. Moreover, the invention provides an implantable system and method for reducing the side effects of pain management therapy on the patient.

Various embodiments of the invention may possess one or more features capable of fulfilling the above objects. Instead of relying upon a patient to provide subjective information relating to sensed pain and then adjusting a pain management system to deliver therapy, the present invention provides an implantable system and method for sensing and analyzing brain wave signals for previously identified brain wave patterns that indicate the presence of pain. Pain management therapy can then be delivered under the control of the implantable system to alleviate the pain, as indicated by the lessening or elimination of the sensed signal associated with pain.

The system and method for pain management therapy of the present invention provides customized signal analysis for each patient's perception of pain. In addition, the pain relief response to the sensed brain wave signals indicating perceived pain can also be customized for each patient. Depletable resources of the system for pain management of the present invention are also more efficiently used, as pain relief therapy is provided only when a need is indicated. More efficient use of the pain relief therapy can also be less traumatic for the patient, in addition to being less demanding on system resources, e.g., battery energy levels, for treating the patient's perceived pain.

Some embodiments of the invention include one or more of the following features: a first lead that includes a first electrode; an implantable signal analyzing unit having the first electrode operatively coupled thereto; an electrical power supply in the implantable signal analyzing unit; a signal analyzer coupled to the electrical power supply, where the signal analyzer receives a brain wave signal through the first electrode and analyzes the brain wave signal to determine the presence of a signal form associated with pain; and a pain management response unit operatively coupled to the sign a analyzer, where the pain management response unit provides therapy for pain management in response to the signal form associated with pain.

The invention involves managing pain through the use of an implantable medical device system by sensing a brain wave signal, analyzing the brain wave signal for an indication of pain, and providing pain management therapy with the implantable medical device system based on the indication of pain from the analysis of the brain wave signal. The brain structure from which the brain wave signal is sensed may or may not be the body structure that receives pain management therapy. Nevertheless, the brain wave signal can continue to be sensed and analyzed to determine whether the pain management therapy is effective in reducing or eliminating the indication of pain in the brain wave signal.

In one embodiment, the present invention provides for a brain wave signal to be sensed from a structure of the brain. The sensed brain wave signal is analyzed to identify selected patterns that are known to be associated with the perception of pain. Pain management therapy from the implantable medical device system is provided based on the identification of the selected patterns in the brain wave signal. Pain management therapy can include delivering electrical stimulation pulses and/or drugs to the patient.

The brain wave signal can continue to be sensed and analyzed by the implantable medical device system during delivery of the pain management therapy. Alternatively, the brain wave signal can be sensed and analyzed by the implantable medical device system after delivery of the pain management therapy. The brain wave signal is analyzed to determine whether the brain wave pattern that indicates perceived pain is still present. Different brain wave signal measurements can be used in identifying a brain wave pattern that indicates perceived pain is still present.

Based on the analysis, parameters used to control the amount of pain management therapy and/or the type of pain management therapy can be adjusted. For example, parameters of electrical pulses delivered to the patient can be changed in response to the brain wave pattern that indicates perceived pain being present. Alternatively, parameters for the amount of drug delivered to the patient can be changed in response to the brain wave pattern that indicates perceived pain being present. These changes can include adjusting parameters that control the amount, duration, and intensity of the pain management therapy. In addition, a hierarchical approach of pain management therapy regimens can be used in addressing the sensed brain wave signal pattern associated with pain.

The above summary of the present invention is not intended to describe each embodiment or every embodiment of the present invention or each and every feature of the invention. Advantages and attainments, together with a more complete understanding of the invention, will become apparent and appreciated by referring to the following detailed description and claims taken in conjunction with the accompanying drawings.

### BRIEF DESCRIPTION OF THE DRAWINGS

FIG. 1 is a flow chart illustrating a technique for feedback control of a pain management system in accordance with the present invention.

FIG. 2 is a schematic diagram illustration an implantable medical device system according to one embodiment of the present invention.

FIG. 3 is a flow chart illustrating a technique for feedback control of a pain management system in accordance with the present invention.

FIG. 4 is a diagrammatic illustration of an EEG of a brain signal illustrating a pain event according to one embodiment of the present invention.

FIG. 5 is a schematic illustration of an implantable medical device system implanted into a patient according to one embodiment of the present invention.

FIG. 6 is a schematic illustration of an implantable medical device system implanted into a patient according to one embodiment of the present invention.

FIG. 7 is a schematic illustration of an implantable medical device system implanted into a patient according to one embodiment of the present invention.

FIG. 8 is a block diagram of the implantable medical device system according to one embodiment of the present invention.

### DESCRIPTION OF THE PREFERRED EMBODIMENTS

The present invention involves techniques for sensing brain wave signals, analyzing the brain wave signals for presence of pain and providing pain management therapy based on the presence of pain from the analysis of the brain wave signals. The brain waves are sensed from any number of locations within the brain, including the sensory thalamus of the brain. The sensed brain wave signals are analyzed for the presence of pain. Features analyzed for the presence of pain include the frequency and the amplitude of the sensed signals. Particular patterns of both the frequency and amplitude of sensed brain waves are known to be associated with pain. Other features of the sensed brain wave may also be used to determine the presence of pain.

In a conventional pain therapy system, the patient is typically relied upon to provide subjective information relating to the sensed pain. Based on the subjective information perceived by the patient, he then adjusts the system to deliver appropriate therapy. This allows for the potential of requesting and receiving more pain therapy than is actually required. As a result, the patient's body may adapt to the over-stimulation, which in turn requires the patient to adjust the system to even higher levels of therapy in order to relieve the pain.

In contrast, the present invention uses an implantable medical device system to identify defined patterns in a sensed brain signal that indicate the presence of pain. Once identified, the implantable medical device system responds by providing pain relief therapy. The system may be programmed to provide a set interval of therapy. After this interval, the system again samples the brain wave signal to determine if the indication of pain is still present in the signal. The system then reactivates the therapy, discontinues the therapy, or changes the therapy type based on the acquired information.

The present invention uses the identified brain signal patterns associated with pain to provide feedback control of an implantable medical device system for pain management. Feedback control of the implantable medical device system for pain management can include, but is not limited to, regulating the application of pain management therapy and regulating the parameters of the applied pain management therapy. These are important improvements in implantable medical device system for pain management.

Regulating the application of the pain management therapy, for example, can reduce the amount of time the system is applying pain management therapy to the patient to only those times when pain signals are sensed. Regulating the pain therapy on/off time may conserve energy, thereby extending the battery life of the implantable system. In addition, regulating the parameters of the pain management therapy delivered to the patient can reduce side effects often seen when pain management therapy is self-administered by the patient or is continuously applied by a pain management system. These side effects include, but are not limited to, tolerance and other causes of loss of efficacy for the pain management therapy.

Pain travels to the brain through different hierarchical structure levels. These hierarchical levels can be broken down into the spinal cord/brainstem, the thalamus, and the cerebral cortex. There is no known discrete center within the brain where pain is recognized. Most regions of the brain have been identified as being involved in the perception of pain. These areas include the sensory and motor cortex areas, the premotor cortex, parietal cortex, frontal cortex, cingulated cortex, insula, and occipital cortex.

Before the signals that cause pain reach the sensory and motor cortex areas, they pass through the thalamus. The thalamus is an egg-shaped structure that lies on the medial-inferior portion of the cerebrum serving as part of the lateral wall of the third ventricle. It includes several different nuclei, most of which send their efferent projections into cerebral cortex. The thalamus is the way station by which virtually all information, including pain, goes to cerebral cortex. Inputs to the thalamus include virtually all levels of the central nervous system, including spinal cord, brain stem, cerebellum, basal ganglia, and cortex. Thus, signals that are perceived as either acute or chronic pain pass through the thalamus and into the cortex of the brain.

The present invention allows for sensing at least one brain wave signal and analyzing the patterns of the brain wave signal for the presence of pain. The sensed brain wave is representative of electrical field potentials generated by the brain. These sensed field potentials provide an electroencephalogram (EEG), referred to as the sensed brain wave signal, which can be analyzed for the occurrence of pain.

In one example, the brain wave signals are preferably sensed from the thalamus. More preferably the brain wave signals are sensed from the sensory nuclei of the thalamus, which is also referred to herein as the sensory thalamus. Alternatively, brain wave signals may be sensed from other nervous system structures that are known to pass pain signals from the body to the brain. These other nervous system structures include, but are not limited to the dorsal horn of the spinal cord, the lateral spino-thalamic tract, the peri-ventricular or peri-aquaductal gray matter of the brain, the sensory and/or motor cortex.

The sensed brain wave signal is then analyzed to identify patterns in the brain wave signal that are indicative of pain. In one example, a brain wave signal sensed from the thalamus that displays a low frequency field potential pattern, defined below, is known to correlate with the patient's reporting of pain. These patterns in a sensed brain wave signal can be used to provide feedback control of an implantable medical device system, as described above and as will be described in greater detail below.

While many of the examples presented below are directed to the detection and treatment of chronic pain, it is recognized that the present invention is not limited only to the detection and treatment of chronic pain. For example, the present invention can be used to detect and treat any number of conditions within the brain. Examples of these conditions include, but are not limited to, migraine headaches, Parkinson's disease, schizophrenia, depression, mania, dystonia, or other neurological disorders where defined patterns are associated with the onset and/or occurrence of the condition.

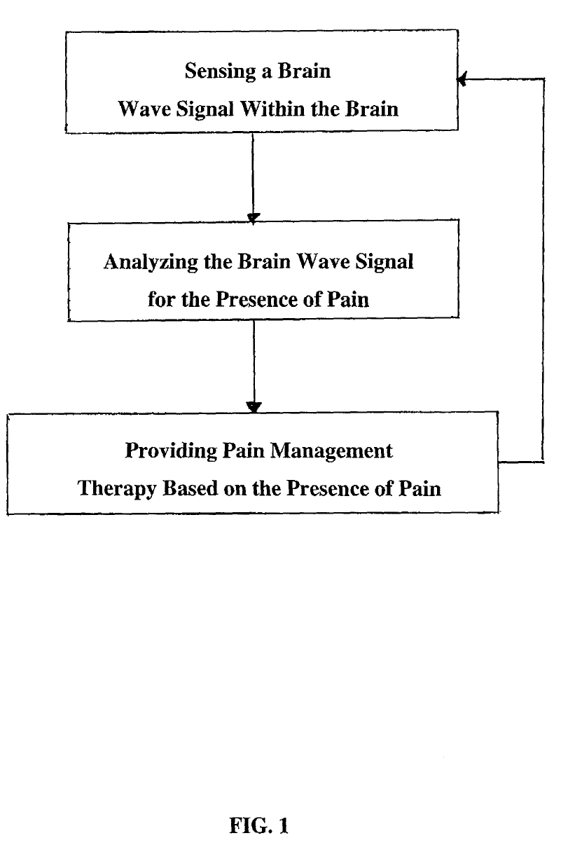

FIG. 1 is a flow diagram illustrating a method of managing pain according to one embodiment of the present invention. At 110, a brain wave signal is sensed within the brain. In one embodiment, a lead having at least a first electrode is implanted into the thalamus, preferably the sensory thalamus. The first electrode can be used to sense a brain wave signal, which is detected as a field potential generated by the brain. The lead can also include additional electrodes for sensing and/or delivery of therapy, as will be described below. Other locations within the brain from which to sense a brain wave signal indicating pain may also exist. These other locations can include, but are not limited to, other nuclei in the thalamus of the brain. Therefore, the exact location within the brain from where the brain signal is sensed may need to be determined for each individual patient.

At 120 of FIG. 1, the sensed brain wave signal is analyzed for the presence of pain with an electronic signal analyzer. In one example, the indication of pain is identified from selected patterns that are known to be associated with the occurrence of pain. For example, a low frequency pattern in a brain wave signal sensed from the sensory thalamus is a selected pattern known to be associated with the perception of pain.

In one embodiment, an example of the low frequency pattern known to be closely associated with the perception of pain includes a brain wave signal sensed from the sensory thalamus and having a frequency field potential of less than about 1 Hz. In an additional embodiment, the low frequency pattern known to be closely associated with the perception of pain includes a brain wave signal sensed from the sensory thalamus and having a frequency field potential of 0.2 to 0.4 Hz. In addition to the low frequency pattern, the amplitude of the brain wave signal is also known to correlate with the intensity of the pain, where the larger the amplitude the greater the perception of pain. See “Thalamic field potentials during deep brain stimulation of periventricular gray in chronic pain” by Nandi et al. Article accepted by PAIN Dec. 13, 2001, Article in Press).

In a further embodiment, a power spectrum might also be used in determining the presence of pain in a sensed brain wave signal. The power spectrum can show and quantitatively characterize the frequency composition of the brain wave signal. After signal conditioning, a Fourier transformation can be made through the use of the Fast Fourier Transform. The result is the power spectrum, i.e., the frequency dependence of the square of the Fourier harmonic amplitudes.

At 130 of FIG. 1, pain management therapy from a pain management response unit is provided based on the analysis and detection of patterns in the brain wave signal that are associated with the perception of pain. In one embodiment, when the presence of pain from the analysis of the brain wave signal is detected, pain relief therapy can include delivering electrical stimulation pulses, having a selected pulse pattern, to the patient. The selected pulse pattern can include a frequency set in a selected frequency range. In addition, the selected pulse pattern can include a voltage set in a selected voltage range. The exact pattern of the selected pulse pattern is determined based in part on the brain structure and/or body area to which the stimuli are delivered to the patient.

In one embodiment, the selected frequency range for the selected pulse pattern includes a programmable value that is 1 pulse per second (PPS) or greater. Alternatively, the selected frequency range for the selected pulse pattern includes a programmable value that is 25 PPS or less. In an additional embodiment, the selected frequency range for the selected pulse pattern includes a programmable value that is 50 PPS or less. The selected frequency range for the selected pulse pattern can also include a programmable value that is 185 PPS or less. In addition, the selected frequency range for the selected pulse pattern can include a programmable value that from 1 to 185 PPS; from 25 to 185 PPS; from 50 to 185 PPS; from 1 to 50 PPS; from 25 to 50 PPS; or from 1 to 25 PPS. The exact frequency required for pain suppression may be determined on a patient-by-patient basis.

Additional parameters for the electrical stimulation pulses may also be programmable. Exact parameter values are specific for the brain structure and/or patient involved. For example, the waveform shape of the pulse can be programmed. Waveform shapes can include, but are not limited to, rectangular, sinusoidal and/or ramped. Other known waveform shapes can also be useful.

The magnitude of each stimulus of the first pulse pattern is also a programmable value of 50 millivolts or more. Alternatively, the magnitude of each stimulus of the first pulse pattern is a programmable value of 10.5 volts or less. Preferably, the magnitude of each stimulus of the first pulse pattern is a programmable value in the range of 50 millivolts to 10.5 volts.

The duration of the each pulse can also be a programmable value. For example, the pulse width of each pulse can be a programmable value of 60 microseconds or greater. Alternatively, the pulse width of each pulse can be a programmable value of 500 microseconds or less. In an additional embodiment, the pulse width of each pulse can be a programmable value of 60 to 500 microseconds.

In one embodiment, the electrical stimulation pulses are delivered to the location where the brain wave signal was sensed. For example, one or more of the same or additional electrodes, discussed in detail below, are used to both sense the brain wave signal and to deliver pain relief therapy. Examples of brain structures where the brain wave signal is sensed and where pain relief therapy is delivered include the thalamus region of the brain, and in particular the sensory thalamus region of the brain.

In an alternative embodiment, the electrical stimulation pulses are delivered to a second location within the brain separate from the location where the brain wave signal is sensed. For example, the brain wave signal can be sensed in the thalamus, while the electrical stimulation pulses are delivered to a region of the brain that is separate from the thalamus. The periventricular gray region of the brain is one example of a region of the brain were electrical stimulation pulses can be delivered for pain management therapy. In an additional example, it is possible to deliver pain management therapy to one or more ventricles of the brain, including, e.g., the third ventricle of the brain. In an additional example, stimulation pulses can be delivered to the motor cortex region of the brain.

In an additional embodiment, electrical stimulation pulses can be delivered to both the location where the brain wave signal for analysis is sensed and one or more secondary locations within the brain. For example, the brain wave signal can be sensed within the sensory thalamus when the second location is the periventricular gray region of the brain. Stimulation pulses can then be delivered to both the sensory thalamus and the periventricular gray region of the brain. Also, it may be possible to provide electrical stimulation pulses for pain management therapy at one or more secondary locations outside of the brain. These locations can include, but are not limited to, locations within the spinal column, a peripheral or cranial nerve, or other known locations used in electrical stimulation pain management therapy systems. Stimulation locations for pain management therapy can also include one or more areas within the brain and one or more areas outside the brain (e.g., spinal cord area).

In addition to delivering electrical stimulation pulses to the brain, the pain management therapy can also include delivering one or more drugs to the body for pain management therapy. Drug delivery for pain management therapy can be provided directly to one or more locations within the brain or to locations outside of the brain. Systemic delivery of drugs for pain management may also be envisioned, although direct infusion of selected amounts of one or more drugs may be preferred. For example, locations within the brain for delivering drugs for pain management therapy include, but are not limited to, the lateral, third or fourth ventricle of the brain. It is also possible to deliver the selected amount of drugs in the sensory thalamus region of the brain and/or in the periventricular gray region of the brain. Other locations within the brain are also possible.

In one example, the implantable system of the present invention can be used to control the operation of one or more drug infusion devices, where each drug infusion device is under the control of a controller, e.g., a microprocessor. For example, sensed signals that indicate perceived pain cause the implantable medical device system of the present invention to control drug delivery from one or more of the drug infusion devices.

Alternatively, the implantable medical device system of the present invention further includes an integrated drug delivery system under the control of a microprocessor. The integrated drug delivery system can then be used alone or in conjunction with the electrical stimulation pulses to provide pain management therapy from the implantable medical device system. Control of the drug delivery device or devices can include, but is not limited to, starting and/or stopping drug delivery through the drug infusion device and/or changing the rate (and therefore the amount of drug) of drug delivery through the drug infusion device. It may also be used to vary the concentration of the drug being delivered by the device (and thereby, the amount of drug received).

Finally, it may be possible to provide electrical stimulation pulses to one or more areas in the brain or other parts of the nervous system while simultaneously delivering pharmacological agents to the brain ventricles, brain tissue, or intrathecal space of the spinal cord. It is also possible to alternate the delivery of drugs and electrical stimulation in any number of patterns and time intervals of delivery. In another embodiment, drug therapy alone could be delivered to the intrathecal space of the spinal cord.

In one embodiment, as the pain management therapy is delivered, the implantable medical device system can continue to analyze the sensed brain wave signal. Alternatively, the pain management therapy is delivered for a set time interval. After the interval, the pain management therapy is discontinued and the sensed brain wave is analyzed for the continued presence of the brain wave pattern that indicates perceived pain. In this way, “chatter” (i.e., noise) in the sensed brain wave signal is avoided.

As mentioned, the brain wave signal can continue to be sensed and analyzed during delivery of the pain management therapy. In one example, the sensed brain wave signal is analyzed for the continued presence of the brain wave pattern that indicates perceived pain during the delivery of drug therapy. Continuous sensing and analysis of the brain wave signal during drug delivery, as compared to when electrical pulses are being used, for pain management therapy is more easily accomplished, as there little or no “chatter” created by the implantable system. In one embodiment, as long as the analysis indicates that the brain wave pattern indicating perceived pain is present, the pain management therapy using drug therapy will continue to be delivered.

Different analysis techniques can be used to determine whether the brain wave pattern that indicates perceived pain is present. For example, one or more thresholds of the amplitude of the brain wave pattern that indicates perceived pain can be used as an indicator when the perceived pain is present or absent for the particular patient. In this situation, the amplitude of the sensed brain wave having the frequency within the range associated with the perception of pain (described above) is analyzed for each patient to determine when the pain is perceived.

This amplitude value, or a selected value just below the amplitude value associated with perceived pain, can be used as the threshold for activating the pain management therapy. In one example, these values for the threshold can be 25 microvolts or higher. Alternatively, the threshold values can be 100 microvolts or less. In addition, the threshold values can be from 25 microvolts to 100 microvolts. Threshold values below or above these values are also possible, as they will be patient dependent.

In one example, when the amplitude value associated with perceived pain exceeds or exceeds the threshold value, the pain management therapy is delivered for a set time interval. The pain management therapy can include either the electrical stimulation pulses and/or drug delivery, as described. After the set time interval expires, the pain management therapy can be discontinued and the brain wave pattern can be analyzed for the presence of the brain wave pattern that indicates perceived pain. In one example, the set time interval during which pain management therapy can be delivered is 10 or more minutes. Alternatively, the set time interval during which pain management therapy can be delivered is for 60 or less minutes The set timer interval for pain management therapy can also be 30 minutes. Preferably, the set time interval for pain management therapy is from 10 to 60 minutes.

After the set time interval expires, the brain wave pattern can be analyzed for the continued presence of the brain wave pattern that indicates perceived pain. Analysis of the brain wave pattern can include measuring and comparing the amplitude of the sensed signals to the threshold values to determine whether the sensed signals meet or exceed a predetermined percentage of the threshold value. In addition, the brain wave pattern can be analyzed to determine how quickly the brain wave pattern that indicates perceived pain returns.

For example, when the brain wave pattern that indicates perceived pain continues to be present the amplitude of the brain wave pattern can be measured and analyzed. When the amplitude of the brain wave is a predetermined percentage or less of the threshold value, therapy will be discontinued. In one example, the predetermined percentage of the threshold value includes fifty (50) percent or less of the threshold value. Alternatively, the predetermined percentage of the threshold value includes from five (5) percent to fifty (50) percent of the threshold value. Preferably, the predetermined percentage of twenty-five (25) percent of the threshold value can be used in the present invention. Therapy is discontinued until a subsequent brain wave pattern that indicates perceived pain reaches the threshold value.

Alternatively, when the brain wave pattern that indicates the perceived pain returns to either the threshold value or a predetermine percentage of the threshold value, the pain management therapy can once again be delivered for the set time interval. For example, when the returning brain wave pattern has an amplitude that meets or exceeds the threshold value then the pain management therapy can once again be delivered. In an additional embodiment, when the returning brain wave pattern has an amplitude that is at least a predetermined percent or greater than the threshold value then pain management therapy can be once again be delivered. For the present embodiment, the predetermined percent is a programmable value of fifty (50) percent or less. Alternatively, the predetermined percent is a programmable value of ten (10) percent or more. Preferably, the predetermined percent is a programmable value of ten (10) percent to fifty (50) percent.

In an alternative embodiment, once the pain management therapy has been delivered for the set time interval, as described above, the pain management therapy is discontinued. The brain wave signal is then sensed and analyzed to determine if the brain wave pattern that indicates perceived pain returns within a predetermined time. In one embodiment, the predetermined time can be set from one (1) minute to one hundred twenty (120) minutes. In one example, the predetermined time can be fifteen (15) minutes.

When the brain wave pattern that indicates perceived pain returns, as described above, any one of the electrical pulse parameters and/or drug delivery parameters (e.g., bolus amount, flow rate, etc.) can be adjusted. For example, the voltage level of the electrical pulses used for pain management therapy can be adjusted when the brain wave pattern indicating perceived pain returns. For example, the electrical pulses used for pain management therapy can be increased by ten (10) percent over an initial voltage setting for the electrical pulses. Alternatively, the electrical pulses used for pain management therapy can be increased by five (5) percent or more over the initial voltage setting for the electrical pulses. Additionally, the electrical pulses used for pain management therapy can be increased by twenty-five (25) percent or more over the initial voltage setting for the electrical pulses. Preferably, the electrical pulses used for pain management therapy can be increased from five (5) percent to twenty-five (25) percent over the initial voltage setting for the electrical pulses. Alternatively, the same percentage increases could be applied to the initial values used for either the pulse widths or frequencies of the electrical pulses.

When the analysis of the brain wave signal indicates that the pattern indicating perceived pain has been eliminated or sufficiently reduced, the pain management therapy can be modified. In one example, pain management therapy is discontinued after a predetermined time once the signal form associated with pain is absent from the brain wave signal. In one embodiment, the predetermined time is a programmable value of at least about 1 minute. Alternatively, the predetermined time is a programmable value of about 15 minutes or less. Alternatively, the predetermined time is a programmable value of about 1 minute to about 15 minutes.

Alternatively, when the analysis of the brain wave signal indicates that the pattern indicating perceived pain has not been eliminated or sufficiently reduced, the patient can continue to receive pain management therapy. In one example, the implantable medical device system can be programmed to continue to deliver the pain management therapy previously provided in the initial pain management therapy. Alternatively, the implantable medical device system can be programmed to adjust the parameters of the pain management therapy and/or move through a hierarchy of different pain management therapy regimens. This feature is important when an initial pain management therapy is unsuccessful in reducing or eliminating the brain wave signal pattern associated with pain.

In one example, a first pain management therapy regimen can be followed by a second pain management therapy regimen in an effort to reduce or eliminate the brain wave signal pattern associated with pain. For example, a first pain management therapy regimen of electrical stimulation pulses to the periventricular gray region of the brain could be followed by a second pain management therapy regimen of electrical stimulation pulses delivered to both the periventricular gray region and the sensory thalamus regions of the brain in an effort to reduce or eliminate the brain wave signal pattern associated with pain.

In an alternative embodiment, the parameters used in the electrical stimulation pulses can be automatically adjusted in an effort to reduce or eliminate the brain wave signal pattern associated with pain. Examples of these modifications were provided above.

Other combinations of pain management therapy can be used in the hierarchy of pain management therapy regimens. For example, the hierarchy of different pain management therapy regimens can have those regimens using drugs to follow regimens that use electrical stimulation pulses. This basic rule would be important in order to conserve a finite drug supply in the implantable medical devices. In addition, the hierarchy of different pain management therapy regimens can have those regimens using two or more sites for delivering electrical stimulation pulses to follow those regimens that use only one site for delivering electrical stimulation pulses. Again, this basic rule would be important to conserve the limited resources (e.g., life of the battery) of the implantable medical device.

One preferred hierarchy of pain management therapy that includes regimens using drugs to follow regimens that use electrical stimulation pulses is as follows. First, an initial regimen of electrical pulse therapy is used. This initial regimen of electrical pulse therapy can include any one of the electrical pulse therapy regimens described for the present invention. When the initial regimen of pain management therapy is not effective, a second regimen of electrical stimulation pulses can be delivered. This second regimen of electrical stimulation pulses can include any one of the modifications previously described. For example, the amplitude of the electrical stimulation pulses can be increased. When the second pain management regimen is not effective, a third pain management regimen can be delivered. The third pain management regimen can include delivering a drug to the patient. In one example, the drug can be a first bolus of 25 microgram of intrathecal Baclofen. Increasing boluses of intrathecal Baclofen can be delivered for subsequent pain management therapy (fourth regimen, fifth regimen, etc.) at thirty minute intervals. So, if a fourth regimen is required, a second bolus of 50 micrograms of intrathecal Baclofen is delivered 30 minutes after the first bolus. If a fifth regimen is required, a third bolus of 75 microgram of intrathecal Baclofen is delivered 30 minutes after the second bolus.

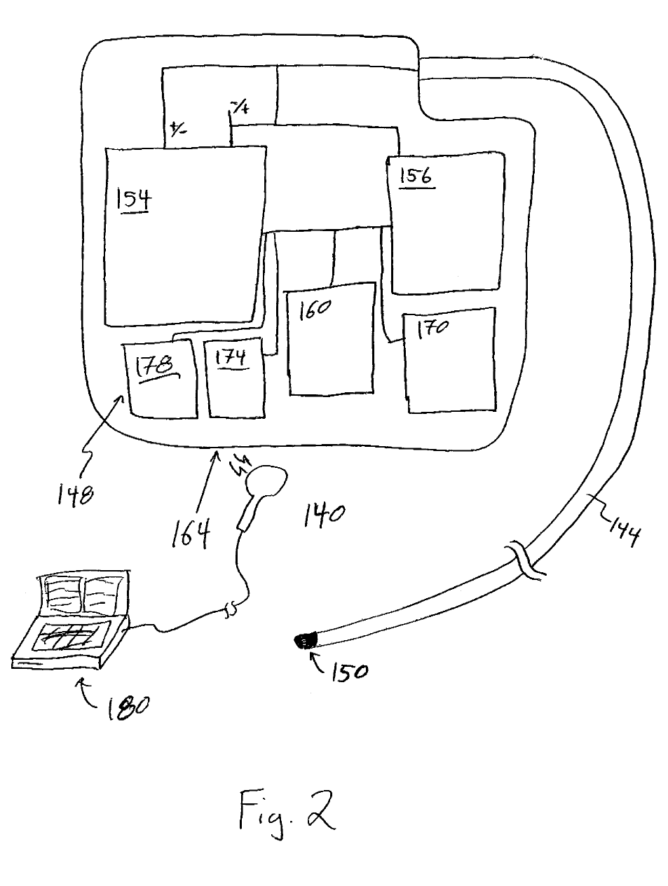

FIG. 2 is a block diagram depicting an example of an implantable medical device system 140. The implantable medical device system 140 includes at least a first lead 144 and an implantable signal analyzing unit 148. The implantable medical device system 140 can also be used to electronically control one or more medical devices (implantable or non-implantable) that are used in addition to the implantable medical device system 140. Other medical devices can include, but are not limited to, implantable pulse generating devices and/or drug pump devices. These aspects of the invention are discussed more fully below.

In the present example, the first lead 144 includes at least a first electrode 150. The first electrode 150 is operatively coupled to the implantable signal analyzing unit 148 to allow for electrical field potentials to be sensed from the brain. Additional electrodes can be included on the first lead 144, where they are used in sensing the brain wave signal. In the present disclosure, a sensed electrical field potential is also referred to as a sensed brain wave signal.

The implantable signal analyzing unit 148 further includes a signal analyzer 154, a pain management response unit 156 and an electrical power supply 160, all of which are preferably hermetically sealed in an implantable housing 164. At least a portion of the implantable housing 164 may be conductive to allow the housing 164 to be used as a pole for sensing the brain wave signal. The signal analyzer 154 is coupled to the electrical power supply 160, and receives the brain wave signal through the first electrode 150. The signal analyzer 154 also analyzes the brain wave signal to determine the presence of the signal form associated with pain.

The pain management response unit 156 is also coupled to the signal analyzer 154 and the electrical power supply 160. The pain management response unit 156 provides therapy for pain management in response to the signal form associated with pain. In one embodiment, the signal analyzer 154 upon determining the presence of the signal form associated with pain in the brain wave signal causes the pain management response unit 156 to provide therapy for pain management.

Therapy for pain management can include, but is not limited to, those therapies or combinations of therapies discussed above. The therapies can include delivering electrical pulses having set pulse parameters to one or more locations within the brain. Alternatively, the electrical pulses can be delivered to one or more locations within the brain and to one or more locations within body outside of the brain. One or more leads, each having one or more electrodes, can be used in delivering the electrical pulses.

In an additional embodiment, the pain management response unit 156 can be used to control one or more drug delivery pump systems. For example, the pain management response unit 156 can be electrically couple to a drug delivery pump system, where electronic control signals from the pain management response unit 156 adjust the amount of drugs pumped from the drug delivery pump system into the patient. Adjusting the amount of drugs pumped from the drug delivery pump system into the patient can include turning the drug pump on or off, adjusting the amount of drug being pumped from the drug delivery pump, or adjusting the concentration of the drug in the drug pump.

The implantable medical device system 140 further includes a controller in the form of, e.g., a microprocessor 170 and memory 174, both of which are operatively coupled to the electrical power supply 160, pain management response unit 156 and the signal analyzer 154. The controller is one form of operatively coupling the pain management response unit 156 and the signal analyzer 154. In one embodiment, the microprocessor 170 is used to execute executable programs stored in memory 174 and either of the pain management response unit 156 and the signal analyzer 154. These programs can include those for analyzing sensed brain wave signals and providing and assessing pain relief therapy delivered to the patient.

In addition, the system 140 may also include a telemetry receiver/transmitter 178 for receiving and transmitting electronic data and/or electronic instructions between the implantable signal analyzing unit 148 and an optional medical device programmer/controller 180.

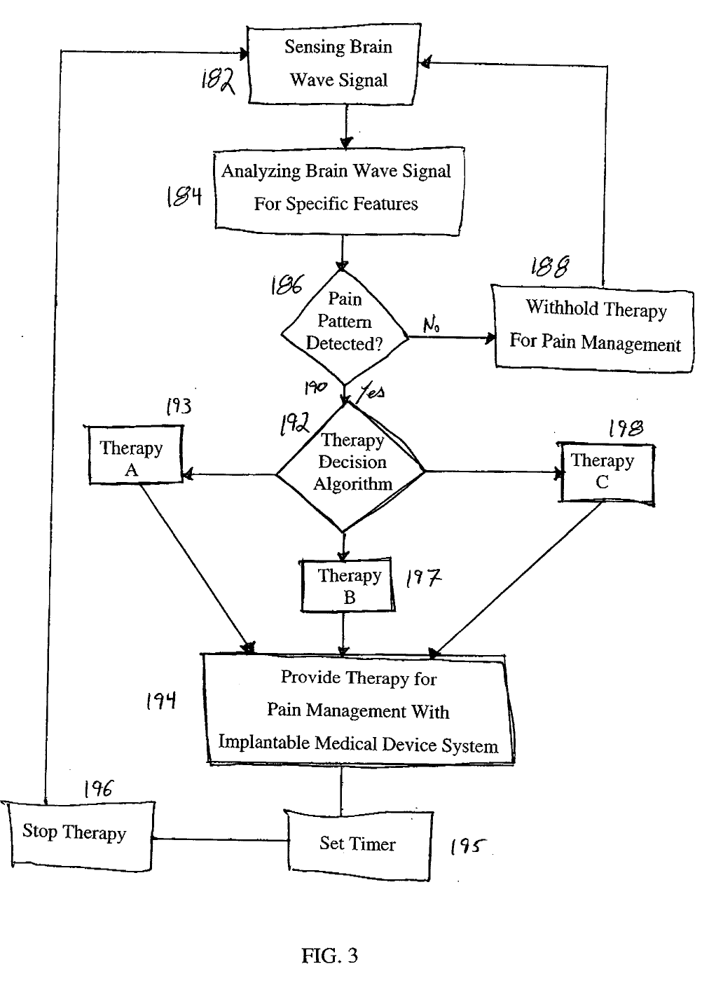

FIG. 3 is a flow chart illustrating one embodiment of the operation of the implantable medical device system for managing pain according to the present invention. At 182, a brain wave signal is sensed from the brain, as discussed. The sensed brain wave signal is then analyzed at 184 for the presence of one or more patterns that are associated with the perception of pain. At 186, a decision is made as to whether patterns that are associated with the perception of pain are detected or not detected.

As mentioned, the presence or absence of a pattern associated with the perception of pain is used as a feedback control for the application of therapy for pain management by the implantable medical device system. For example, when no pattern that is associated with the perception of pain is present in the brain wave signal, the therapy for pain management is withheld from the patient 188. If, however, a pattern that is associated with the perception of pain is present in the brain wave signal, the implantable medical device system proceeds to a process for providing therapy for pain management via 190.

At 192, a therapy decision algorithm is used to assess how to respond to the detected pattern associated with the perception of pain. In one example, the therapy decision algorithm can utilize one or more of the decision pathways described above for determining the pain management therapy when a brain wave pattern that indicates perceived pain is detected.

For example, the frequency and the amplitude of the brain wave signal can be used to determined the presence of the perceived pain. A first pain management therapy 193 can then be provided to the patient at 194. A timer at 195 can be used to measure the set time interval over which the first pain management therapy is delivered. In one embodiment, the timer measures the set time interval over which the pain management therapy is delivered, as described above. Once the timer at 195 expires, the pain management therapy can be discontinued at 196. The brain wave signal is then sensed and analyzed again at 182 and 184. The decision is then made as to whether patterns that are associated with the perception of pain are detected or not detected at 186.

In one embodiment, the decision at 186 as to whether patterns that are associated with the perception of pain are detected or not detected are as described above. For example, brain wave signals having the predetermined percentage or greater of the threshold value indicates that additional therapy needs to be provided. Alternatively, when the brain wave pattern indicating the perceived pain returns to, or is greater than, the threshold value, pain management therapy will be continued. In an additional embodiment, when the brain wave pattern that indicates perceived pain returns within a predetermined time, the pain management therapy will be continued.

When a pattern that is associated with the perception of pain is present in the brain wave signal at 186, the implantable medical device system proceeds again to the process for providing therapy for pain management via 190. This second encounter with the therapy decision algorithm 192 can cause a different pain management therapy regimen to be delivered. Examples of these pain management regimens that are different than the initial pain management therapy regimen are provided above.

At 192, the therapy decision algorithm is used to determine the next response to the continued detected pattern associated with the perception of pain. In one example, the therapy decision algorithm can utilize a second pain management therapy 197, and deliver the therapy to the patient at 194. The second pain management therapy 197 can be part of a programmed hierarchy of pain management therapies, as discussed above.

The timer at 195 can be used to measure the set time interval over which the second pain management therapy is delivered at 194. Once the timer at 195 expires, the pain management therapy can be discontinued at 196. The brain wave signal is then sensed and analyzed again at 182 and 184. The decision is then made as to whether patterns that are associated with the perception of pain are detected or not detected at 186. If necessary, the therapy decision algorithm 192 can be used to determine a third pain management therapy at 198 that is then delivered to the patient at 194. This overall process can be repeated until all the levels of pain management therapy have been delivered to the patient and/or the patterns that are associated with the perception of pain are not detected at 186.

In one specific example, the brain wave signal is sensed in the thalamus at 182. The sensed brain wave signal is then analyzed at 184 for the presence of one or more patterns that are associated with the perception of pain. In the present example, the brain wave sensed in thalamus includes an amplitude that is greater than or equal to 25 microvolts and includes a frequency in the range of 0.2 to 0.7 Hz. Based on these specific features, the decision is made at 186 that this brain wave pattern is associated with the perception of pain.

At 192, the therapy decision algorithm determines that the first pain management therapy regimen 193 is to be delivered to the patient at 194. In the present example, the first pain management therapy regimen 193 includes providing electrical stimulation pulses that have one (1) volt with a pulse width of 120 microseconds and at a frequency of 5 pulses per second. These pulses can be delivered to the periventricular gray matter of the brain. The electrical stimulation pulses of the first pain management therapy are delivered over a fifteen (15) minute interval that is timed by timer 195.

Once the timer 195 expires and the therapy is discontinued at 196, the brain wave signal is once again sensed and analyzed at 182 and 184. The analysis of the brain wave signal after the first pain management therapy regimen has been delivered may or may not be for identifying the same signal features that caused the system to initially deliver the first pain management therapy regimen. In the present example, the brain wave signals are analyzed to determine their relative amplitude compared to the amplitude of the brain wave signal that indicated the need for the first pain management therapy regimen. When the sensed brain wave signals have an amplitude of fifty (50) percent or greater of the amplitude of the brain wave signal that indicated the need for the first pain management therapy regimen, there is a pain detected pattern at 186.

The therapy decision algorithm 192 proceeds to the second pain management therapy regimen 197, which is delivered to the patient at 194. In the present example, the second pain management therapy regimen 197 includes providing electrical stimulation pulses having an increased voltage. For example, the new voltage value can be one and one-quarter (1.25) volts with the pulse width of 120 microseconds and at the frequency of 5 pulses per second. These pulses can be delivered to the same region where the brain wave signal was sensed (i.e., the thalamus). The electrical stimulation pulses of the second pain management therapy are delivered over the fifteen (15) minute interval that is timed by timer 195. It is understood that two or more of the pulse parameters and/or the time of delivery for the second pain management therapy regimen could have been adjusted.

Once the timer 195 expires and the therapy is discontinued at 196, the brain wave signal is once again sensed and analyzed at 182 and 184. The analysis of the brain wave signal after the second pain management therapy regimen has been delivered may or may not be for identifying the same signal features that caused the system to continue to the second pain management therapy regimen. In the present example, the brain wave signals are once again analyzed to determine their relative amplitude compared to the amplitude of the brain wave signal that indicated the need for the first pain management therapy regimen. When the sensed brain wave signals have an amplitude of fifty (50) percent or greater of the amplitude of the brain wave signal that indicated the need for the first pain management therapy regimen, there is a pain detected pattern at 186.

The therapy decision algorithm 192 proceeds to the third pain management therapy regimen 198, which is delivered to the patient at 194. In the present example, the third pain management therapy regimen 198 includes providing electrical stimulation pulses having an increased pulse width. For example, the pulse width can be doubled from 120 microseconds to 240 microseconds. These pulses can be delivered to the same region as before (i.e., the periventricular gray). The electrical stimulation pulses of the third pain management therapy are delivered over the fifteen (15) minute interval that is timed by timer 195. It is understood that two or more of the pulse parameters and/or the time of delivery for the second pain management therapy regimen could have been adjusted.

For the above example, if it were determined that the relative amplitude of the sensed brain wave signal had an amplitude of less than fifty (50) percent of the amplitude of the brain wave signal that indicated the need for the pain management therapy regimen, then the subsequent pain management therapy would be withheld at 188. The brain wave signal would continue to be sensed and analyzed at 182 and 184, and a determination as to whether the pain pattern is detected made at 186.

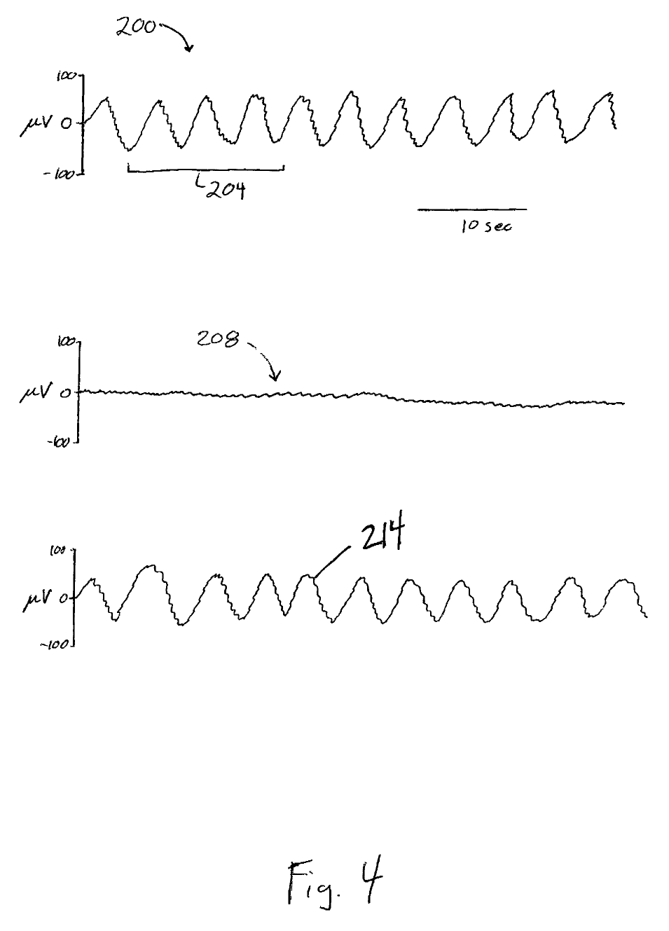

FIG. 4 is an illustration of brain wave signal field potentials recorded from the sensory thalamus. The brain wave signal field potentials include a first signal 200. The first signal 200 includes the low frequency pattern 204 associated with the perception of pain. As previously discussed, this low frequency pattern 204 may be a brain wave signal sensed from the sensory thalamus that has a frequency field potential of less than about 1 Hz, and includes frequency field potential of 0.2 to 0.4 Hz.

The brain wave signal field potentials of FIG. 4 also include a second signal 208. The second signal 208 illustrates a field potential recording from the sensory thalamus during stimulation of the periventricular gray region of the brain with the selected pulse pattern. As the second signal 208 shows, in the amplitude of the low frequency pattern is reduced as compared to the first signal 200. This reduced amplitude low frequency pattern is known to be associated with a lessening in the level of pain perceived by the patient. As previously discussed, the selected pulse pattern may preferably include a programmable value that is less, e.g., than 50 pulses per second, where the exact frequency required for pain suppression will be patient dependent. In addition, it is possible that more than one selected pulse pattern can be effective in reducing or eliminating the low frequency pattern associated with the perception of pain.

A third signal 214 is shown in FIG. 4. The third signal 214 illustrates a field potential recording from the sensory thalamus during stimulation of the periventricular gray region of the brain with a pulse pattern that has a frequency of at least 50 PPS. As the third signal 214 shows, the low frequency pattern 204 associated with the perception of pain is present during the stimulation of the periventricular gray region. This illustrates that the frequency at which the stimulation pulses are delivered to the periventricular gray region of the brain may be an important aspect in the therapy for pain management according to the present invention.

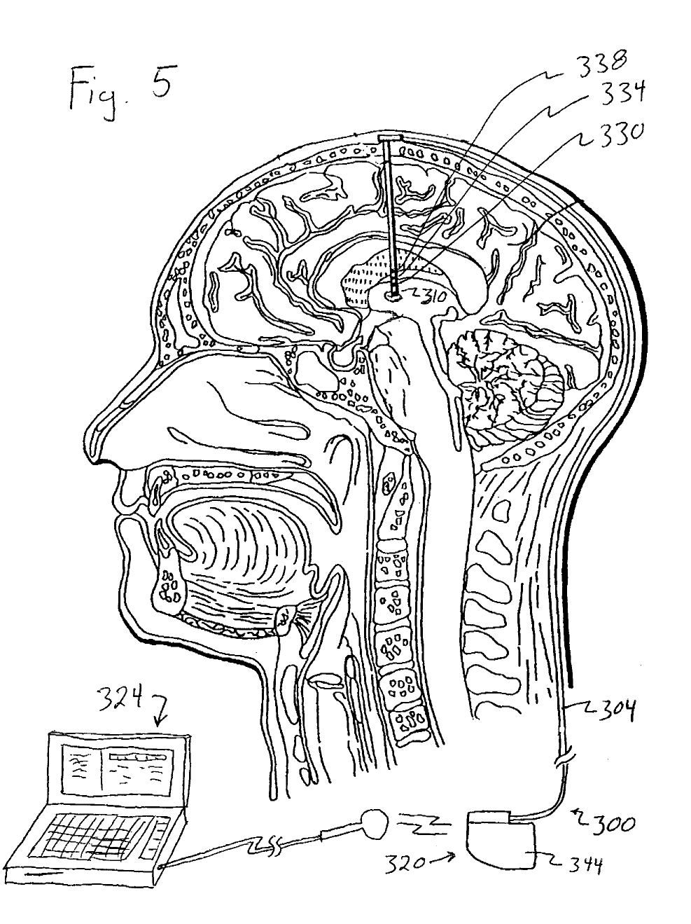

FIG. 5 shows one embodiment of an implantable medical device system 300 according to the present invention. In one embodiment, portions of system 300 can be implanted below the skin of a patient. The system includes generally at least a first lead 304 having at least a first electrode 310 implantable in a structure of a brain. Electrode 310 can serve to sense a brain wave signal (e.g., a electrical field potential) from the structure of the brain under the control of the implantable signal analyzer unit 320. The implantable signal analyzer unit 320 can also use electrode 310 to deliver therapy for pain management in response to a signal form associated with pain.

In the present example, the therapy for pain management includes electrical stimuli delivered to the structure of the brain through the first electrode 310 under the control of the implantable signal analyzing unit 320. In an alternative embodiment, additional electrodes are included on the first lead 304 and are implanted in the brain structure to sense the brain wave signal and to deliver therapy for pain management under the control of the implantable signal analyzing unit 320.

First electrode 310 can be implanted in any one or more structures of the brain, as previously described. In the example shown in FIG. 3, first electrode 310 are coupled to the implantable signal analyzing unit 320 through the first lead 304. First electrode 310 can take the form of a device capable of detecting nerve cell or axon activity. In one embodiment, first electrode 310 is located in any one or more structures of the thalamus of the brain, as previously described. A medical device programmer/controller 324 may also be used to communicate and program the implantable signal analyzing unit 320. In one embodiment, the medical device programmer/controller 324 transmits and receives data from the implantable signal analyzing unit 320 to communicate with the implanted pulse generator through a telemetry link. Such telemetric systems may use, for example, radio frequency, ultrasound, infrared, or other like communication means.

In one embodiment, the first lead 304 further includes a second electrode 330, a third electrode 334, and a fourth electrode 338, where the first electrode 310, the second electrode 330, the third electrode 334, and the fourth electrode 338 are operatively coupled to the implantable signal analyzing unit 320. The electrodes 310, 330, 334, and 338 on the first lead 304 serve not only to deliver the therapy for pain management, but also to receive the brain wave signal. Each electrode 310, 330, 334, and 338 may be individually connected to the implantable signal analyzing unit 320 through the first lead 304. Depending upon the situation, one or more stimulation/sensing leads with any number of electrodes may be used. For example, lead model 3387 DBS™ sold by Medtronic, Inc. of Minneapolis, Minn. may be used. Additional useful sensing and stimulation lead models include models 3389 DBS™ and 3388 DBS™, also sold by Medtronic, Inc.

The implantable signal analyzing unit 320 includes an electrical power supply, a signal analyzer, and a pain management response unit, encased in an implantable housing 344. In one embodiment, the implantable housing 344 is a hermetically sealed housing. The signal analyzer receives a brain wave signal through at least the first electrode 310 and analyzes the brain wave signal to determine the presence of a signal form associated with pain, as previously discussed. In addition, the signal analyzer can be used to receive the brain wave signal through any combination of the first electrode 310, second electrode 330, third electrode 334, and fourth electrode 338.

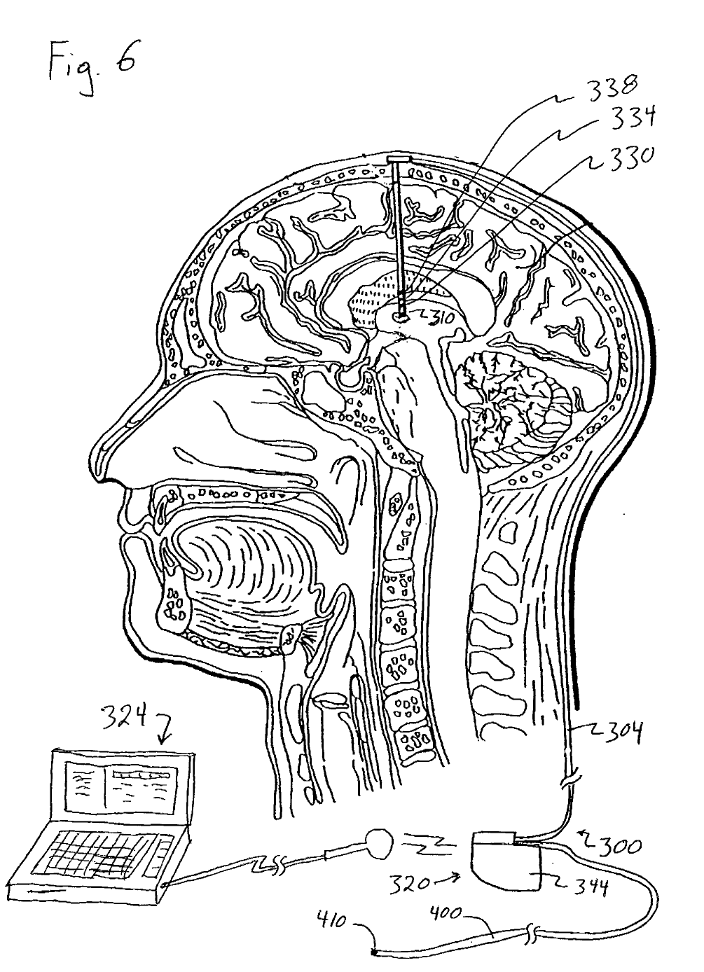

FIG. 6 shows an additional embodiment of the implantable medical device system 300 according to the present invention In addition to the features of the implantable medical device system 300 described above, the system 300 further includes a second lead 400. The second lead 400 includes at least one stimulation electrode 410 that may be implantable. In one example, the second lead 400 is implantable in a structure of a brain, as previously described. Structures of the brain can include, but are not limited to, the periventricular gray matter, motor cortex, and/or the sensory cortex. In an additional embodiment, the second lead 400 is implantable in a location outside the brain, as previously discussed. These locations can include, but are not limited to, the epidural space of the spinal cord, the intrathecal space of the spinal cord, on or near a peripheral nerve, and/or on or near a cranial nerve.

The second lead 400 is operatively coupled to pain management response unit in the implantable signal analyzing unit 320. The implantable signal analyzer unit 320 can use the stimulation electrode 410 to deliver therapy for pain management in response to a detected signal form associated with pain. In one embodiment, the pain management response unit may deliver series electrical pulses in two locations, as discussed above, through at least the first electrode 310 on the first lead 304 and the stimulation electrode 410 on the second lead 400.

The stimulation electrode 410 can take the form of a device capable of delivering electrical pulses to either the brain or other structure of the body. In one embodiment, stimulation electrode 410 is located in any one or more structures of the cortex of the brain, including the periventricular gray region of the brain, as previously described. Alternatively, the stimulation electrode 410 is located in the epidural space of the spinal cord, the intrathecal space of the spinal cord, on or near a peripheral nerve, and/or on or near a cranial nerve. The second lead 400 can also include additional electrodes that are operatively coupled to the implantable signal analyzing unit 320. Examples of the second lead include, but are not limited to, lead models 3387 DBS™, 3389 DBS™ and 3388 DBS™, model 3487A Pisces Quad® lead, model 3888 Pisces Quad Plus® lead, model 3887 Pisces Quad Compact® lead, model 3587A Resume II® lead, model 3982 SymMix® lead, and model 3987 On-Point® lead, all of which are sold by Medtronic, Inc. of Minneapolis, Minn.

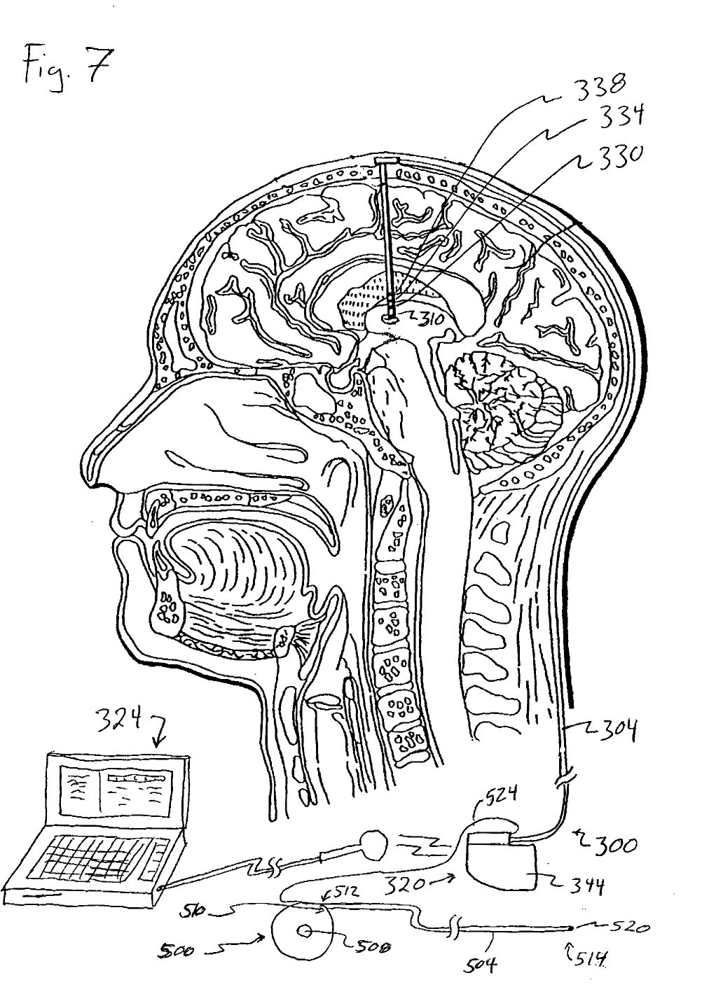

FIG. 7 shows an additional embodiment of the implantable medical device system 300 according to the present invention In addition to the features of the implantable medical device system 300 described above, the system 300 further includes a electronic drug infusion pump system 500 and a drug infusion catheter 504 that is operatively coupled to the electronic drug infusion pump system 500. In one embodiment, the electronic drug infusion pump system 500 is operatively coupled to the pain management response unit in the implantable medical device system 300. This allows the electronic drug infusion pump system 500 to be electronically controlled by the implantable medical device system 300.

As shown in FIG. 7, the electronic drug infusion pump system 500 may be implanted below the skin of the patient. The system 500 may include a port 508 through which a drug can be delivered to the system 500. The drug is delivered from the system 500 to the patient through a catheter port 510 into the drug infusion catheter 504. The drug infusion catheter 504 can be positioned in the body, as previously described, to allow delivery of the drug to the body under the control of the pain management response unit. An example of a drug delivery system is found in U.S. Pat. No. 6,263,237 (Rise) assigned to Medtronic, Inc., Minneapolis, Minn., which is incorporated by reference. An additional examples of drug delivery systems include model 8628 and 8627 SynchroMed® Programmable Infusion Systems sold by Medtronic, Inc. of Minneapolis, Minn.

In one embodiment, the drug infusion catheter 504 includes a proximal end 512 and a distal end 514. The proximal end 512 can be releasably coupled to the catheter port 510 to allow for drugs to be pumped through a lumen (not shown) in the catheter 504 to an opening 520 in the catheter 504. The distal end 514 can be implanted into the body, including regions of the brain and spinal cord, as previously discussed.

The distal end 514 of the catheter 504 can include a rounded profile for minimized tissue disruption during insertion. The opening 520 is positioned at or adjacent the distal end 514 of the catheter 504 to allow for delivering the drug to the body. In one embodiment, the opening includes a microporous portion that allow for infusion and filtering of the drug. Alternatively, the opening can further include a valve mechanism for controlling the flow of the drug through the catheter 504.

The electronic drug infusion pump system 500 further includes a drug reservoir that is accessible through port 508, a pump coupled to the drug reservoir, and an electronic pump control for controlling the pump. In one embodiment, a control lead 524 couples the electronic drug infusion pump system 500 and the implantable signal analyzing unit 320. The control lead 524 allows the implantable signal analyzing unit 320 to control the electronic pump control of the pump system 500. In one embodiment, the implantable signal analyzing unit 320 can control the electronic pump control of the pump system 500 to allow drugs to be administered to the body through the drug infusion catheter 504 as part of the therapy for pain management, as described above. In addition, the delivery of drugs can also be combined with the series of electrical pulses as part of the therapy for pain management. Alternatively, the stimulator and drug pump systems could be integrated into a single implantable unit to both stimulate and deliver drugs.

Alternatively, the implantable medical device system 320 and the electronic drug infusion pump system 500 could be built into a single unit having all of the features of each device. This device would eliminate the need for the control lead 524.

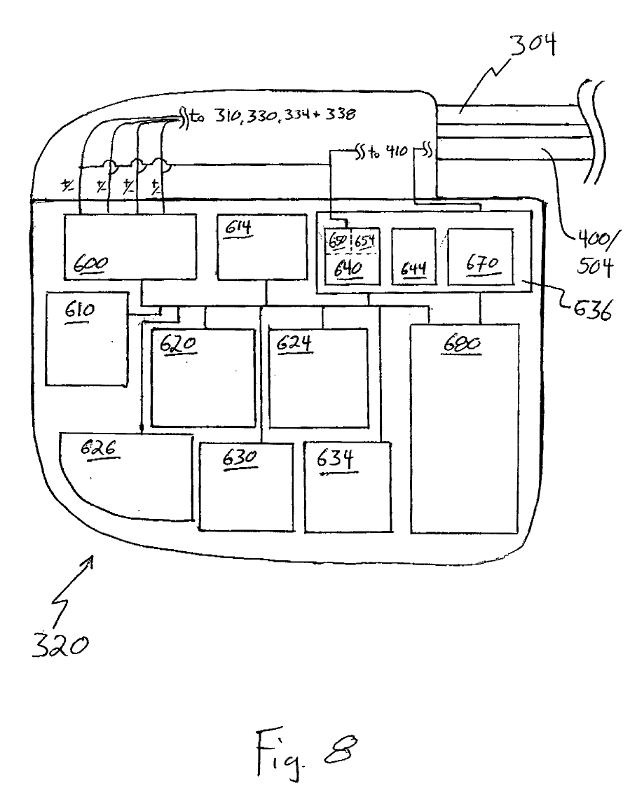

FIG. 8 is a block diagram depicting one embodiment of an implantable signal analyzing unit 320 of the present invention in greater detail. A brain wave signal sensed with one or more of the electrodes 310, 330, 334, and/or 338 may be amplified and/or filtered by amplifier 600 and filter 610, respectively. The brain wave signal can then be converted to a digital representation by analog to digital converter 614. The brain wave signal may then be further processed by a signal analyzer 620 or may be input to a microprocessor 624 for processing. The implantable signal analyzing unit also further includes an electrical power supply 626 and optional communication components 630 to allow for telemetry communication between the implantable signal analyzing unit 320 and the medical device programmer/controller 324.

In one embodiment, the signal analyzer 620 is used to analyze the brain wave signal received through one or more of electrodes 310, 330, 334, and/or 338 and to determine the presence of a signal form associated with pain, as previously discussed. In one embodiment, analyzing the brain wave signal and determining the presence of the signal form associated with pain is accomplished through the use of an algorithm stored in a memory 634. The algorithm can be embodied ,e.g., as program code retrieved from memory 634 and executed by signal analyzer 620 and/or microprocessor 624.

In one embodiment, the signal analyzer 620 measures the field potential of the brain wave signal sensed through one or more of electrodes 310, 330, 334, and/or 338. The signal analyzer 620 analyzes the brain wave signal for selected patterns that are known to be associated with the perception of pain. As previously discussed, a low frequency pattern of less than 1 Hz in a brain wave signal sensed from the sensory thalamus is a selected pattern known to be associated with the perception of pain. In one embodiment, the signal analyzer 620 compares the sensed brain wave signal to stored parameters of brain wave signals (e.g., frequency and amplitude of the brain wave signal) that have been determined to cause the perception of pain for the patient. When the pattern known to be associated with the perception of pain is detected, the signal analyzer 620 causes a pain management response unit 636 to deliver therapy for pain management.

The signal analyzer 620 and the microprocessor 624 may both be coupled to the pain management response unit 636. The pain management response unit 636 provides therapy for pain management in response to the signal analyzers 620 identification of patterns in the brain wave signal that are associated with pain. In one example, the pain management response unit 636 includes a pulse generator 640 capable of generating a series of electrical pulses for the therapy for pain management. The series of electrical pulses can be delivered at a frequency as described above so as to reduce or eliminate the brain wave signal pattern associated with the perception of pain.

In addition to delivering the series of electrical pulses, other pain therapies are also possible (e.g., use of drug or a combination of drug and electrical pulses) as described above. In one embodiment, the series of electrical pulses can then be delivered to one or more of the electrodes 310, 330, 334, and/or 338. In an additional embodiment, the series of electrical pulses can then be delivered to at least the stimulation electrode 410 located on the second lead 400. Alternatively, the series of electrical pulses can then be delivered to a combination of one or more of the electrodes 310, 330, 334, and/or 338 and at least the stimulation electrode 410.

The pain management response unit 636 also includes a timer 644. The timer 644 is capable of timing the delivery of therapy for the pain management, as described, for a predetermined time interval after the signal analyzer 620 determines the presence of the signal form associated with pain. As previously discussed, the set time interval during which therapy can be delivered is a programmable value. The signal analyzer 620 may or may not continue to analyze the sensed brain wave signal for the presence of the signal form associated with the perception of pain as the timer 644 counts the set time interval. For example, during drug delivery the signal analyzer 620 can continue to sense and analyzed the brain wave. However, during delivery of electrical pulses, the signal analyzer 620 does not analyze sensed brain wave signals, for the reasons previously discussed.

When the set time interval of timer 644 expires, the brain wave signal can be sensed and analyzed, as described. The pain management response unit 636 can then modifies the therapy for pain management when the signal analyzer 620 determines the continued presence of the signal form associated with the perception of pain, as discussed above. In an additional embodiment, the pain management response unit 636 can utilize the hierarchy of modifications to the therapy for pain management, as discussed above, when the timer 644 expires and the signal analyzer 620 determines the continued presence of the signal form associated with pain. Alternatively, when the signal analyzer 620 determines that the signal form associated with the perception of pain is no longer present in the brain wave signal, the pain management response unit 636 can then withhold the therapy for pain management.

The signal analyzer 620 analyzes the sensed brain wave signal during the therapy for pain management is being delivered. The signal analyzer 620 analyzes the brain wave signal for the presence of the signal form associated with pain. While the signal form associated with pain is detected, the signal analyzer 620 will continue to provide pain relief therapy through the use of the pain management response unit 636. As discussed, the signal analyzer 620 can be programmed to apply a hierarchy of pain relief therapy depending upon the effectiveness of each programmed pain relief regimen.

The patterns and/or parameters of the brain wave signal associated with the perception of pain can be identified and programmed into the implantable signal analyzing unit 320 at the time the unit 320 is implanted in the patient. Initially, one or more of the leads 304 and 400, and/or catheter 504 are implanted into the body. In one example, sterotactic implantation techniques are used to implant one or more the leads 304 and 400, and/or catheter 504 into the brain of the patient into one or more of the locations previously discussed.

The leads and/or catheter are coupled to the implantable signal analyzing unit 320. A brain wave signal is sensed with one or more of the electrodes of lead 304, as discussed. The brain wave signal is analyzed to identify one or more low frequency patterns that are associated with pain and stored in memory 630. This can be accomplished by sensing the brain wave signal with the implantable medical device system 300, where the brain wave signal is displayed on the medical device programmer/controller 324.

The medical device programmer/controller 324 can be used to cause the implantable signal analyzing unit 320 to deliver the series of electrical pulses through one or more of the implanted electrode, as discussed. As the series of electrical pulses are delivered, the amplitude of the low frequency brain wave signal is analyzed as the patient provides feedback on their perception of pain. When there is a positive correlation between the reduction in the perception of pain by the patient and a reduction in the amplitude of low frequency patterns in the sensed brain wave, then the low frequency patterns in the sensed brain wave are confirmed to be associated with the perception of pain. These patterns and/or their characteristics (e.g., the frequency and amplitude of the signal) can then be stored in memory 634 and used by the signal analyzer 620 in the analysis and identification of a signal form associated with pain.

The pulse generator 640 may also include a frequency range selector 650 and/or a voltage range selector 654. The frequency range selector 650 can be used to set the frequency of the series of electrical pulses delivered for pain relief therapy, as discussed above, and the voltage range selector 654 can be used to set the voltage of the series of electrical pulses delivered for pain relief therapy. The values for both the frequency range selector 650 and the voltage range selector 654 can be set under the control of the pain management response unit 636. Depending on the number of false positives or false negatives the could be manually adjusted or system may self-adjust the therapy. Additional therapy techniques and processes could be added to the implantable signal analyzing unit 320 for treating the patient. For example, therapies under the control of the implantable signal analyzing unit 320 could include stimulation of the brain to control tremors and other symptoms of Parkinson's disease, and other movement disorders and/or the delivery of drugs to the spinal cord to control spasticity.

At the time the present invention is implanted within the patient, the clinician may program certain key parameters into the memory 634 of the device or may do so later via telemetry. These parameters may be updated subsequently as needed. Alternatively, the clinician may elect to use default values. The clinician ordinarily will program the range of values for the pulse width, amplitude and frequency of the series of electrical pulses used for pain relief therapy. The clinician can adjust the parameters of the electrical pulses via telemetry with a medical device programmer. In order to assess the occurrence of pain, the sensed brain wave signals can be stored in memory 634 over time, and retrieved by telemetry for assessment by the physician. The physician can use the stored data to reset therapy or monitoring characteristics in implantable signal analyzing unit 320.

In an additional embodiment, the implantable signal analyzing unit 320 can include a drug pump controller 670. The drug pump controller 670 may be coupled to and controlled by the pain management response unit 634. In one example, the drug pump controller 670 is used to control an electronic drug infusion pump system, such as system 500 of FIG. 5. In this situation, a control lead can be used to operatively couple the implantable signal analyzing unit 320 and the electronic drug infusion pump system. The electronic drug infusion pump system can deliver a set amount of drugs. Alternatively, the sensed brain wave signal is involved in the feedback loop system to control the amount of drugs supplied based on the analysis of the sensed brain wave signal. As discussed, the catheter for drug delivery can be located in the brain and/or other location within the body. In addition, the function of the catheter could be integrated into a lead used for delivering the series of electrical pulses for pain management therapy (e.g., the lead could have a lumen for drug delivery). In either case, both the drug delivery and the electrical pulses could occur in the same area of the body, which may lead to a synergistic improvement in the treatment of pain.

In an alternative embodiment, the capability for drug delivery is contained within the implantable signal analyzing unit 320. For example, the pain management response unit 634 further controls an electronic drug infusion pump 680 housed within the implantable signal analyzing unit 320. The electronic drug infusion pump 680 is capable of providing drugs for the therapy for pain management, as previously discussed. An outlet of the electronic drug infusion pump 680 is coupled to a lumen in the drug infusion catheter 504, which is attached to the implantable signal analyzing unit 320.

The preceding specific embodiments are illustrative for the practice of the invention. It is to be understood, therefore, that other expedients known to those skilled in the art or disclosed herein, may be employed without departing from the invention or the scope of the appended claims. For example, the present invention is not limited to using identifiable pain signals in the feedback control of an implantable medical device. The present invention is also not limited to using the implantable medical device in the control of pain, per se, but may find further application in identifying and treating migraine headaches, Parkinson's disease, schizophrenia, depression, mania, or other neurological disorders where predetermined patterns associated with the condition can be identified in one or more sensed brain wave signals. The present invention further includes within its scope methods of making and using systems and/or apparatus for carrying out the methods described hereinabove.

What is claimed is:

1. An implantable medical device system comprising:
a first lead, comprising a first electrode; and
an implantable signal analyzing unit, wherein the first electrode is operatively coupled to the implantable signal analyzing unit, the implantable signal analyzing unit comprising:
an electrical power supply;
a signal analyzer coupled to the electrical power supply, wherein the signal analyzer is operable to receive a brain wave signal through the first electrode and is operable to analyze the brain wave signal to determine the presence of a signal form associated with pain; and
a pain management response unit operatively coupled to the signal analyzer, where the pain management response unit is operable to provide therapy for pain management in response to the signal form associated with pain.

2. The implantable medical device system of claim 1, wherein the pain management response unit comprises a pulse generator operatively coupled to the first electrode, the pulse generator capable of delivering a series of electrical pulses through the first electrode.

3. The implantable medical device system of claim 2, wherein the pulse generator comprises a frequency range selector.

4. The implantable medical device system of claim 2, wherein the pulse generator comprises a voltage range selector.

5. The implantable medical device system of claim 1, wherein the first lead further comprises a second electrode, a third electrode and a fourth electrode, wherein the first electrode, the second electrode, the third electrode, and the fourth electrode are operatively coupled to the implantable signal analyzing unit.

6. The implantable medical device system of claim 5, wherein the signal analyzer is operable to receive the brain wave signal through the first electrode and one or more of the second electrode, the third electrode, and the fourth electrode.

7. The implantable medical device system of claim 5, wherein the therapy for pain management provided by the pain management response unit includes a series of electrical pulses delivered through one or more of the first electrode, second electrode, the third electrode, and the fourth electrode.

8. The implantable medical device system of claim 1, the system further comprising a second lead that comprises a stimulation electrode, wherein the stimulation electrode is operatively coupled to the pain management response unit, whereby the pain management response unit is capable of delivering a series electrical pulses through the stimulation electrode.

9. The implantable medical device system of claim 8, wherein the pain management response unit comprises a pulse generator, whereby the pain management response unit is capable of delivering a series of electrical pulses through the first electrode and the stimulation electrode.

10. The implantable medical device system of claim 1, wherein the pain management response unit comprises a drug infusion catheter operatively coupled to an electronic drug infusion pump system, the electronic drug infusion pump system operatively coupled to the pain management response unit, whereby the pain management response unit is capable of activating the electronic drug infusion pump system to provide a drug through the drug infusion catheter for the therapy for pain management.

11. The implantable medical device system of claim 10, wherein the pain management response unit comprises a pulse generator capable of providing a series electrical pulses through the first electrode as a part of the therapy for pain management.

12. The implantable medical device system of claim 10, wherein the first lead further comprises a second electrode, a third electrode, and a fourth electrode operatively coupled to the implantable signal analyzing unit, wherein the signal analyzer is operable to receive the brain wave signal through one or more of the first electrode, second electrode, the third electrode, and the fourth electrode, and wherein the pain management response unit is capable of delivering a series electrical pulses through one or more of the first electrode, second electrode, the third electrode, and the fourth electrode for the pain management therapy.

13. The implantable medical device system of claim 1, wherein the signal analyzer is capable of analyzing the brain wave signal during the therapy for pain management for the presence of the signal form associated with pain.

14. The implantable medical device system of claim 1, wherein the pain management response unit comprises a timer capable of timing delivery of therapy for pain management for a predetermined time interval after the signal analyzer determines the presence of the signal form associated with pain.

15. The implantable medical device system of claim 14, wherein the pain management response unit is operable to modify the therapy for pain management when the timer expires and the signal analyzer is operable to determine a continued presence of the signal form associated with pain.

16. The implantable medical device system of claim 15, wherein the pain management response unit is operable to utilize a hierarchy of modifications to the therapy for pain management when the timer expires and the signal analyzer is operable to determine the continued presence of the signal form associated with pain.

17. An implantable signal analyzing unit, comprising:
an electrical power supply;
a signal analyzer coupled to the electrical power supply, wherein the signal analyzer is operable to receive a brain wave signal and is operable to analyze the brain wave signal to determine the presence of a signal form associated with pain; and
a pain management response unit operatively couple to the signal analyzer, where the pain management response unit is operable to provide therapy for pain management in response to the signal form associated with pain.

18. The implantable signal analyzing unit of claim 17, wherein the pain management response unit comprises a pulse generator capable of generating a series of electrical pulses for the therapy for pain management.

19. The implantable signal analyzing unit of claim 18, wherein the pulse generator comprises a frequency range selector.

20. The implantable signal analyzing unit of claim 18, wherein the pulse generator comprises a voltage range selector.

21. The implantable signal analyzing unit of claim 17, wherein the pain management response unit comprises an electronic drug infusion pump that is capable of providing a drug for the therapy for pain management.

22. The implantable signal analyzing unit of claim 21, wherein the pain management response unit comprises a pulse generator capable of generating a series of electrical pulses for the therapy for pain management.

23. The implantable signal analyzing unit of claim 17, wherein the signal analyzer is capable of analyzing the brain wave signal during the therapy for pain management for the presence of the signal form associated with pain.

24. The implantable signal analyzing unit of claim 17, wherein the pain management response unit comprises a timer capable of timing delivery of therapy for pain management for a predetermined time interval after the signal analyzer determines the presence of the signal form associated with pain.

25. The implantable signal analyzing unit of claim 24, wherein the pain management response unit is operable to modify the therapy for pain management when the timer expires and the signal analyzer is operable to determine a continued presence of the signal form associated with pain.

26. The implantable signal analyzing unit of claim 25, wherein the pain management response unit is operable to utilize a hierarchy of modifications to the therapy for pain management when the timer expires and the signal analyzer is operable to determine the continued presence of the signal form associated with pain.

27. A method of managing pain, comprising:
sensing a brain wave signal within a brain with an implantable medical device system;
analyzing the brain wave signal for an indication of pain with the implantable medical device system; and
providing pain management therapy with the implantable medical device system based on the indication of pain from the analysis of the brain wave signal.

28. The method of claim 27, comprising positioning a first electrode in a sensory thalamus region of a brain, and sensing the brain wave signal through the first electrode of the implantable medical device system.

29. The method of claim 28, wherein providing pain management therapy comprises delivering electrical stimulation pulses having a frequency in a selected frequency range to the sensory thalamus region of the brain with the implantable medical device system.

30. The method of claim 29, wherein providing pain management therapy comprises delivering electrical stimulation pulses in a selected voltage range with the implantable medical device system.

31. The method of claim 29, wherein providing pain management therapy comprises delivering electrical stimulation pulses having the frequency in the selected frequency range to both the sensory thalamus region and the periventricular gray region of the brain with the implantable medical device system.

32. The method of claim 28, wherein providing pain management therapy comprises delivering electrical stimulation pulses having a frequency in a selected frequency range to a periventricular gray region of the brain with the implantable medical device system.

33. The method of claim 28, wherein providing pain management therapy comprises delivering a drug to a body with the implantable medical device system.

34. The method of claim 33, wherein delivering the drug comprises delivering the drug directly to the sensory thalamus region of the brain with the implantable medical device system.

35. The method of claim 33, wherein delivering the drug comprises delivering the drug directly to a periventricular gray region of the brain with the implantable medical device system.

36. The method of claim 33, wherein providing pain management therapy comprises delivering electrical stimulation pulses having a frequency in a selected frequency range to the sensory thalamus region of the brain with the implantable medical device system.

37. The method of claim 33, wherein providing pain management therapy comprises delivering electrical stimulation pulses having a frequency in a selected frequency range to a periventricular gray region of the brain with the implantable medical device system.

38. The method of claim 33, wherein providing pain management therapy comprises delivering electrical stimulation pulses having the frequency in the selected frequency range to both the sensory thalamus region and a periventricular gray region of the brain with the implantable medical device system.

39. The method of claim 27, wherein providing pain management therapy comprises analyzing the brain wave signal during pain management therapy with the implantable medical device system for the brain wave signal that provides the indication of pain.

40. The method of claim 27, comprising timing the pain management therapy provided with the implantable medical device for a predetermined time interval after the indication for the indication of pain.

41. The method of claim 40, comprising modifying the pain management therapy provided with the implantable medical device when the predetermined time interval expires and the analysis of the brain wave signal indicates pain.

42. The method of claim 41, wherein modifying the pain management therapy provided with the implantable medical device comprises utilizing a hierarchy of modifications to the therapy for pain management when the time interval expires and the analysis of the brain wave signal indicates pain.
